(19) United States
(12) Patent Application Publication
(10) Pub. No.: US 2015/0320384 A1
(43) Pub. Date: Nov. 12, 2015

(54) Methods and Systems for Detecting an Object in a Subject with Ultrasound

(71) Applicant: University of Washington, Seattle, WA (US)

(72) Inventors: Bryan Cunitz, Seattle, WA (US); Wei Lu, Seattle, WA (US); Neil Owen, Bothell, WA (US); Oleg A. Sapozhnikov, Seattle, WA (US); Michael R. Bailey, Seattle, WA (US); Lawrence A. Crum, Bellevue, WA (US); John Kucewicz, Bellevue, WA (US); Barbrina Dunmire, Seattle, WA (US); Adam Maxwell, Seattle, WA (US); Mathew D. Sorensen, Seattle, WA (US)

(21) Appl. No.: 14/705,730

(22) Filed: May 6, 2015

Related U.S. Application Data
(60) Provisional application No. 61/989,376, filed on May 6, 2014.
(60) Provisional application No. 61/989,386, filed on May 6, 2014.

(51) Int. Cl. A61B 8/08; A61B 8/00; A61B 8/14

(52) U.S. Cl. CPC A61B 8/085; A61B 8/14; A61B 8/5276; A61B 8/5223; A61B 8/0875; A61B 8/0841; A61B 8/0891; A61B 8/5253; A61B 8/461

(57) ABSTRACT

A system and method for detecting, via ultrasound, a concretion in a subject are provided. One or more ultrasound pulses are transmitted into the concretion and at least one object of interest, such as a bubble, present in the concretion. Reflection signals from the concretion and the bubble are then contrasted using the twinkling artifact, and a filter removes motion signals. An output device, such as a display, provides an indication of the presence of the concretion based on the reflection signals.

### CROSS-REFERENCE TO RELATED APPLICATIONS

This application claims priority to U.S. Provisional Patent Application Ser. No. 61/989,376 filed on May 6, 2014, and to U.S. Provisional Patent Application Ser. No. 61/989,386 filed on May 6, 2014, both of which are hereby incorporated by reference in their entirety.

### GOVERNMENT RIGHTS

This invention was made with government support under Grant No. DK043881, awarded by the National Institutes of Health and Grant No. SMST003402, awarded by the National Space Biomedical Research Institute. The government has certain rights in the invention.

### BACKGROUND

Kidney stones are one of the most common and painful urological disorders around the world, and global prevalence and incidence of kidney stones is believed to be increasing. The lifetime incidence of kidney stones is about 13% in men and 7% in women. Additionally, once an individual has formed a stone, the likelihood of recurrence is about 35-50% within five years and up to 80% at 10 years.

Abdominal X-ray and computerized tomography (CT) are common diagnostics used for kidney stones in a subject. However, X-ray and CT expose a subject to radiation, which is associated with various health effects.

Ultrasound is another imaging modality that can be used to image a kidney stone in a patient, and does not pose a risk of radiation exposure. Additionally, ultrasound is inexpensive relative to CT, portable, and widely available. However, ultrasound is currently limited due to factors such as a broad range of sensitivity and specificity.

An ability to improve kidney stone detection using ultrasound may result in greater adoption of ultrasound for the management of kidney stones.

### SUMMARY

In accordance with the present invention, a system and a method are defined for detecting an object in a body of a subject.

In one embodiment, the method may comprise transmitting one or more ultrasound pulses to the object and to at least one object of interest on the object, and receiving one or more reflection signals, wherein the one or more reflection signals comprise reflection signals corresponding to the one or more ultrasound pulses reflected from the object and reflection signals corresponding to the one or more ultrasound pulses reflected from the at least one object of interest on the object. The method may further comprise contrasting individual reflection signals from the one or more reflection signals, displaying a magnitude of interpulse variability, removing, via a filter, motion signals, and causing an output device to provide an indication of the presence of the object based on the reflection signals corresponding to the one or more ultrasound pulses reflected from the at least one object of interest on the object.

In one embodiment, the object may be a kidney stone, a gall stone, a calcification, a crevice, a crack, a calculus, a foreign object, or an ossification, and the tissue may be a kidney tissue, a fatty tissue, a bone, or a cyst.

The method may further comprise applying color or another indication, such as an X or drawing a shape like a circle, to locations above a threshold brightness on an image that provides the indication of the presence of the object.

In one embodiment, the reflection signals corresponding to the one or more ultrasound pulses reflected from the at least one object of interest comprise variability in phase or amplitude. The phase and amplitude between pulses may be random and not deterministic as with motion of the target tissue or object.

Deterministic means not random; for example, blood flow is predictable and not random. As the target, a particular packet of cells in a blood flow moves toward the transducer each successive pulse in the ensemble travels a shorter round trip distance to the packet and back. The travel time is predictable and the therefore so is the change in phase between the pulses. Such a blood flow represents a constant or slowly changing velocity, and thus Doppler shows the blood flow as one color representing that velocity. The effect of small bubbles oscillating randomly on the stone is to create random changes in phase and amplitude among pulses in the ensemble, resulting in the Doppler showing a constantly changing mosaic of color.

In one embodiment, artificially amplifying or attenuating at least one of the one or more reflection signals via a filter accentuates variability in phase or amplitude.

In another embodiment, a method to diagnose, prognose, or monitor a kidney stone in a subject is provided. The method may comprise transmitting one or more ultrasound pulses to the object and to at least one object of interest on the object, and receiving one or more reflection signals, wherein the one or more reflection signals comprise reflection signals corresponding to the one or more ultrasound pulses reflected from the object and reflection signals corresponding to the one or more ultrasound pulses reflected from the at least one object of interest on the object. The method may further comprise contrasting individual reflection signals from the one or more reflection signals, displaying a magnitude of interpulse variability, removing, via a filter, motion signals, and causing an output device to provide an indication of the presence of the object based on the reflection signals corresponding to the one or more ultrasound pulses reflected from the at least one object of interest on the object.

In another embodiment, a system for measuring an object within a body of a subject is provided. The system comprises an ultrasound transducer and a physical computer readable storage medium. The ultrasound transducer is used to acquire images of the living tissue and the object. The physical computer readable storage medium comprises instructions executable to perform functions including transmitting one or more ultrasound pulses to the object and to at least one object of interest on the object and receiving one or more reflection signals, wherein the one or more reflection signals comprise reflection signals corresponding to the one or more ultrasound pulses reflected from the object and reflection signals corresponding to the one or more ultrasound pulses reflected from the at least one object of interest on the object. The functions may further comprise contrasting individual reflection signals from the one or more reflection signals, displaying a magnitude of interpulse variability, removing, via a filter, motion signals, and causing an output device to provide an indication of the presence of the object based on the reflection signals corresponding to the one or more ultrasound pulses reflected from the at least one object of interest on the object.

The methods and system may be used to detect a kidney stone. The system and method may be used to diagnose, provide a prognosis, monitor, and guide treatment decisions for a kidney stone in a subject.

The system and method may be used for a subject having a concretion within a tissue, including but not limited to nephrolithiasis. The nephrolithiasis may include any type of kidney or urinary stone. The system and method may be used to inform and aid in making a determination whether a subject is likely to require surgery to remove the kidney stone, monitor the kidney stone, and make a treatment decision based on a prognosis related to use of the system and method.

These as well as other aspects and advantages of the synergy achieved by combining the various aspects of this technology, that while not previously disclosed, will become apparent to those of ordinary skill in the art by reading the following detailed description, with reference where appropriate to the accompanying drawings.

### BRIEF DESCRIPTION OF THE FIGURES

FIG. 1 depicts a schematic of an exemplary system in accordance with at least one embodiment;

FIG. 2 depicts a simplified flow diagram of an example method that may be carried out to detect an object in a body of a subject, in accordance with at least one embodiment;

FIG. 3 depicts an experimental setup for an evaluation of kidney stone detection using the twinkling artifact, in accordance with at least one embodiment;

FIG. 4 depicts a table comprising various optimization parameters, in accordance with at least one embodiment;

FIG. 5 depicts a graph plotting SNR over number of cycles per burst for both the stone and the glass sphere, in accordance with at least one embodiment;

FIG. 6 depicts a graph plotting SNR over a Doppler transmit angle for both the stone and the glass sphere, in accordance with at least one embodiment;

FIG. 7a depicts a graph plotting SNR over Pulse Repetition Frequency (PRF), in accordance with at least one embodiment;

FIG. 7b depicts a graph plotting SNR over voltage, in accordance with at least one embodiment;

FIG. 7c depicts a graph plotting SNR over ensemble length, in accordance with at least one embodiment;

FIG. 8 depicts a table 800 of preliminary clinical results, in accordance with at least one embodiment;

FIG. 9a depicts an example frame illustrating a B-mode ultrasound image in accordance with at least one embodiment; and

FIG. 9b depicts an example frame illustrating a twinkling artifact ultrasound image, in accordance with at least one embodiment.

### DETAILED DESCRIPTION

In the following detailed description, reference is made to the accompanying figures, which form a part thereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

For the present application, the term “stone” may mean any piece of calculus material that may be found in an organ, duct, or vessel of a subject, including stones, stone fragments, and stone dust that may result from the application of shock waves or other therapeutic procedures. A pulse is an acoustic wave of a certain duration. The excitation of one or more elements in a transducer probe generates a pulse.

Kidney stones are often observed on B-mode ultrasound as a bright spot. The brightness is due to the large impedance mismatch between the kidney stone and surrounding tissue. However, other structures within the image can also appear as bright spots.

Kidney stones may be observed to “twinkle” when viewed under color Doppler ultrasound. Doppler ultrasound is a mode of ultrasound imaging to detect blood flow, and color Doppler is a technique that estimates the average velocity of flow within a vessel by color coding the information. The direction of blood flow is assigned the color red and blue, indicating flow toward or away from the ultrasound transducer. Color Doppler can be used to overlay color on a B-mode ultrasound image. An algorithm may prevent color from being added on an object that is bright. Blood is expected to be dark in the ultrasound image and the system looks to mark flowing blood with color. Alternatively, instead of or in addition to color, another indication such as an X or a drawing a shape such as a circle, for example, may be added to the image. B-mode, or ray-line, is made of narrow beams, wherein the width of the beam and the width of overlap between beams determines the transverse resolution. In another type of B-mode, plane wave B-mode, a focus may be directed distal or not at all, and the goal is to create a plane wave broader than the stone. The goal with plane-wave is to have uniform excitation across a stone, for example to create a uniform shadow behind the stone.

Thus, although a Doppler method is applied, parameters are changed from standard Doppler and additions are provided, as discussed herein.

Traditionally, the twinkling artifact (TA) is the random display of color that can intermittently appear in the presence of kidney stones in color Doppler mode. The twinkling may be caused by small bubbles on the stone surface. The bubbles may be micron-sized gas pockets on hydrophobic stone surface regions. Multiple bubbles trapped in cracks or crevices can oscillate from a strong incident wave, such as a Doppler pulse. Since Doppler comprises multiple pulses in a burst or ensemble, the initial pulse excites the bubbles to oscillate, which alters the reflection of later pulses. Doppler processing, e.g., autocorrelation within the ensemble, detects regions or locations in the image where there is variability among the pulses in the ensemble. Doppler places color in those locations unless the location also has a high-reflected signal corresponding to bright in the image. If the brightness is above a threshold defined by system algorithms, the write priority decides to show the brightness and not overlay the color. In any event, the variability within the ensemble tricks the Doppler processing to place color either on the stone or around the brightest parts of the stone. The evidence of this is that bubbles cause twinkling and some of the methods described herein are devised to affect a bubble mechanism. However, there are some other factors that also may possibly cause twinkling and the etiology of twinkling is not fully understood. Bubbles may not be the only mechanism for twinkling in all cases. Many of the methods described herein still utilize a twinkling artifact effect in the signals to identify and detect the stone in the image regardless of the root cause of the interpulse variability.

In one embodiment, detection ultrasound is used to located stones within an organ, duct, or vessel such as the kidney, wherein the ultrasound waves reflected from a stone are preferentially selected and displayed using the twinkling artifact relative to ultrasound waves reflected off blood and tissue.

In one embodiment, the bright spots in an ultrasound image are located and then used to identify a stone. In another embodiment, areas in an ultrasound image with large interpulse variability are located and then to used identify as a stone. Doppler ultrasound may then be used to apply color on portions of the ultrasound image that are also bright. This is in contrast to current Doppler ultrasound, which prevents color (the indication of flow) from being placed on bright objects or portions of the ultrasound.

### 1. Overview

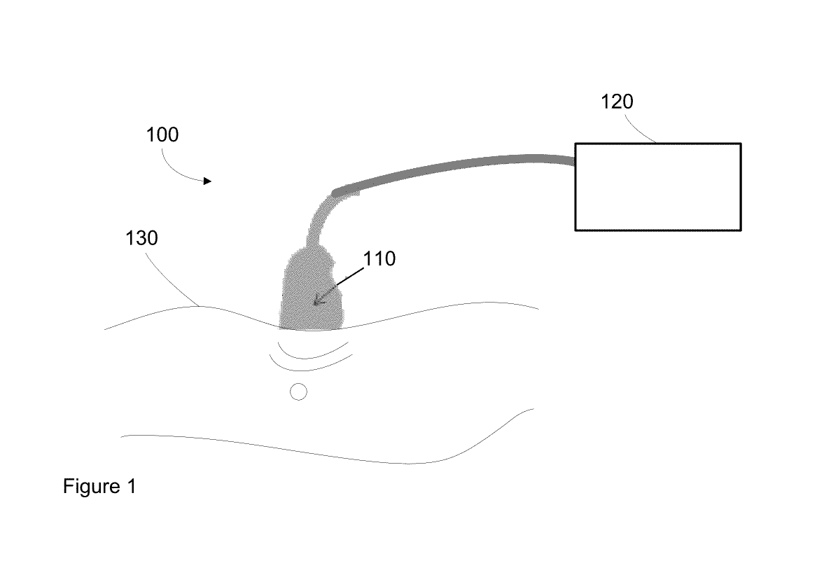

FIG. 1 depicts a schematic of an exemplary system 100 in accordance with at least one embodiment. The system 100 may be used, among other things, to measure an object within a body of a subject. Thus, the system 100 may be used on a subject in vivo. As referenced herein, a subject may be a human subject.

In FIG. 1, an ultrasound system is shown as system 100. The system 100 may include a transducer 110 and a computing system 120. A sample 130 to be imaged is also shown in FIG. 1.

The transducer 110 may have a fixed or a variable focal length. The transducer 110 may be a linear, curvilinear, or phased array and be able to steer the beam to image the stone off the axis of the transducer 110.

The computing system 120 may include a processor, data storage, and logic. These elements may be coupled by a system or bus or other mechanism. The processor may include one or more general-purpose processors and/or dedicated processors, and may be configured to perform an analysis on the output from the ultrasound system. An output interface may be configured to transmit output from the computing system to a display.

Doppler ultrasound often uses a black and white B-mode image to show the anatomy based on the strength of reflections form reflectors or scatters in the tissue. B-mode imaging uses individual elements to direct the acoustic energy to a narrow beam by focusing acoustic rays lines. Resolution is enhanced at the user-selectable focus, at the sacrifice of pre- and post-focal resolution. In one example embodiment, B-mode imaging may be used to determine what might be a kidney stone as B-mode imaging has a high level of sensitivity, and the twinkling artifact may be used to verify the kidney stone, as the twinkling artifact has a high level of specificity.

In one alternative embodiment, flash or plane wave imaging may be used instead of ray line B-mode imaging.

In another example embodiment, either no compression or reverse compression of the brightness in an image is applied, to make the bright and the dark stand out. The combination of a bright spot with a proximal dark shadow can be used together to indicate a stone. This information can be used in combination with other information provided herein to indicate a stone. This technique can be called a voting scheme, so for example if three of the four approaches indicate a stone is present, then the system marks the object or region as a stone.

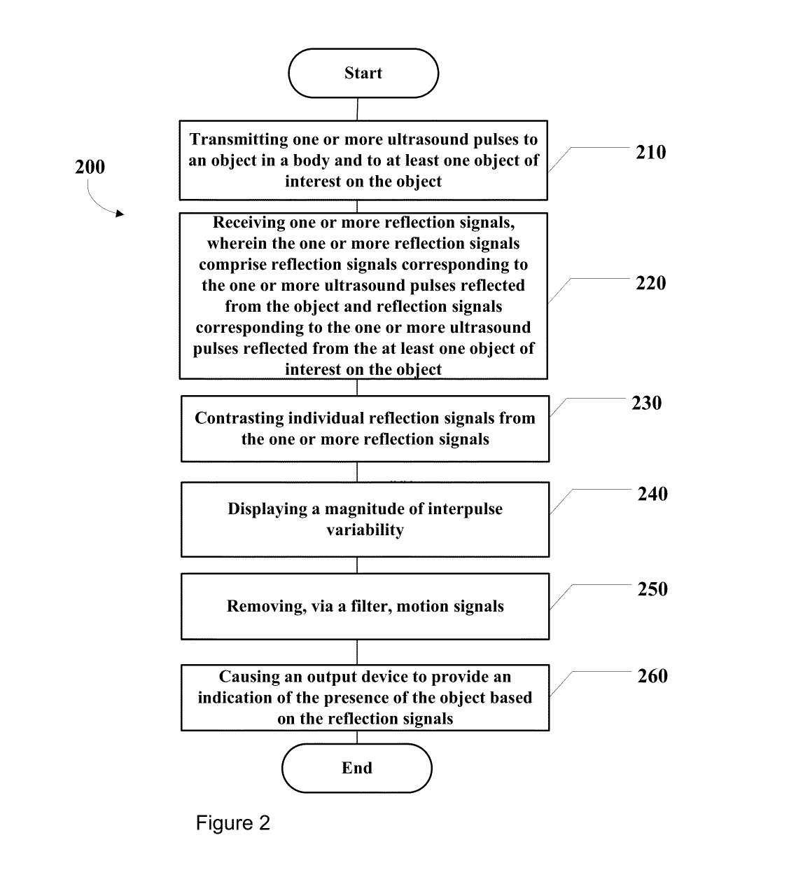

FIG. 2 depicts a simplified flow diagram of an example method that may be carried out to determine an object in a body of a subject, in accordance with at least one embodiment. Method 200 shown in FIG. 2 presents an embodiment of a method that, for example, could be used with the system 100.

In addition, for the method 200 and other processes and methods disclosed herein, the flowchart shows functionality and operation of one possible implementation of the present embodiments. In this regard, each block may represent a module, a segment, or a portion of program code, which includes one or more instructions executable by a processor for implementing specific logical functions or steps in the process. The program code may be stored on any type of computer readable medium, for example, such as a storage device including a disk or hard drive. The computer readable medium may include a physical and/or non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, a tangible storage device, or other article of manufacture, for example. Alternatively, program code, instructions, and/or data structures may be transmitted via a communications network via a propagated signal on a propagation medium (e.g., electromagnetic wave(s), sound wave(s), etc.).

The method 200 allows for imaging and determining an object, such as a concretion or kidney stone, using ultrasound. An ultrasound system may be the same or similar to the system 100 of FIG. 1. The method 200 may be used to diagnose, prognose, or monitor treatment for a kidney stone in a subject.

Initially, the method 200 includes transmitting one or more ultrasound pulses to the object and to at least one object of interest on the object, at block 210. In operation, a subject is positioned at a designated location to allow for observation of desired biological tissues and concretion of the sample 130. The sample 130 may be observed in vivo, as shown in the example depicted in FIG. 1.

A transducer probe, such as the transducer 110 of FIG. 1, delivers one or more ultrasound pulses into the body. An ultrasound pulse is generally in the frequency range of about 1 to 5 megahertz, and travels through one or more tissues in the body. In one example embodiment, the transducer is positioned on the body to deliver an ultrasound pulse through tissues of a kidney. However, the transducer may be positioned on the body to deliver one or more pulses through different tissues, such as the liver or gallbladder, for example. The liver, gall bladder, and pancreas all can have stones, as can the salivary glands. Because salivary glands are small and superficial, the appropriate imaging range would be much higher, for example in the range of 5-20 MHz.

The ultrasound pulses may be delivered to the body in a Doppler ensemble. A Doppler ensemble comprises a number of pulses per cycle, and a number of cycles per burst. An initial excitation pulse may be different in the number of cycles, frequency, and amplitude. In one example embodiment, the number of bursts in an ensemble ranges from 2-20. The amplitude for the pulses sent in the ensemble comprises the same amplitude.

The method 200 then includes receiving one or more reflection signals, wherein the one or more reflection signals comprise reflection signals corresponding to the one or more ultrasound pulses reflected from the object and reflection signals corresponding to the one or more ultrasound pulses reflected from the at least one object of interest on the object, at block 220.

The ultrasound pulses travel as waves and hit a boundary between tissues, at which point some of the waves are reflected back to the transducer, while some travel further on until they reach another boundary and are reflected. Signals from the reflected waves may be received by the transducer and may be relayed to the computing device, such as the computing device 120.

The method 200 includes contrasting individual reflection signals from the one or more reflection signals, at block 230. Reflected pulses in the ensemble may be compared and contrasted by autocorrelation or Doppler processing to reveal regions of high interpulse variability or Doppler power

The method then includes displaying a magnitude of interpulse variability, at block 240. Once the areas of high variability are identified in the image, the system may use a method to make them apparent to the viewer such as but not limited to adding a color to the location potentially corresponding to the magnitude of the variability or Doppler power.

The method 200 then includes removing, via a filter, motion signals, at block 250.

In one example embodiment, the filter may be high to cut out the blood flow that the Doppler typically searches for.

The method 200 includes causing an output device to provide an indication of the presence of the object based on the reflection signals corresponding to the one or more ultrasound pulses reflected from the at least one object of interest on the object, at block 260.

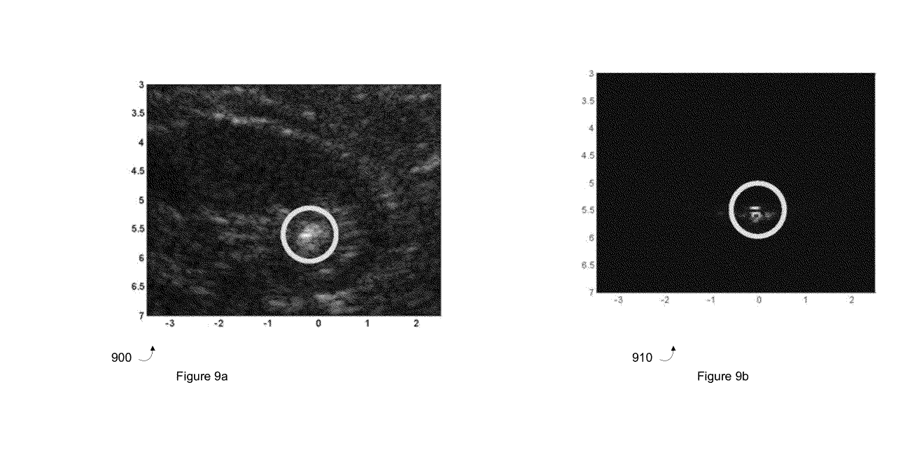

A computing system, such as the computing system 120, may execute instructions to show the results on a display. In one example embodiment, the indication of the presence of the object is displayed by overlying a color indicator on the image. In another example embodiment, the brightness is enhanced. An example of an output image is depicted in FIG. 9b, further described below.

### 2. Example Embodiments

An evaluation was performed to detect kidney stones in vitro applying the twinkling artifact in combination with a Doppler imaging sequence. A V-1 Verasonics Data Acquisition System was programmed and controlled via a host computing device, such as the computing device 120 of FIG. 1. The computing device used MATLAB R2011b and was programmed to work with the ATL HDI C5-2 ultrasound imaging probe.

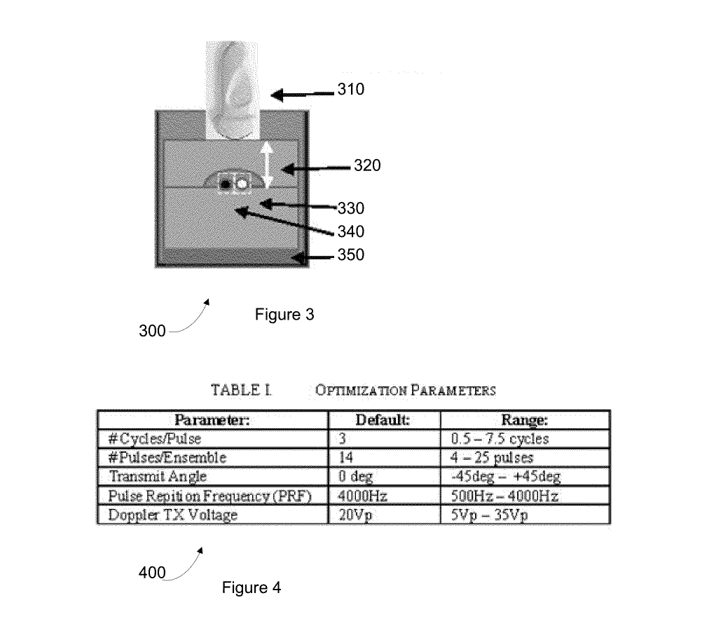

FIG. 3 depicts an example system 300 for an evaluation of kidney stone detection using the twinkling artifact, in accordance with at least one embodiment. The example system 300 comprises an abdominal imaging probe 310, an agar-glycerol phantom 320, a kidney stone 330, a glass sphere 340, and an acoustic absorber 350.

The agar-glycerol phantom 320 was an agar-glycerol based soft-tissue mimicking phantom having 5 cm of material between the probe and the targets, and a 1 cm fluid filled void around the targets, which was then sandwiched with 4 cm of material and the acoustic absorber 350 on the bottom to prevent reflections.

The kidney stone 330 was a 4 mm kidney stone extracted from a kidney stone patient, and the glass sphere 340 was a 4 mm glass sphere; the kidney stone 330 and the glass sphere 340 were used as targets.

The abdominal imaging probe 310 was aligned with the targets such that the brightest hyperecho from both targets was achieved in a B-mode scan. The glass sphere 340 was used as a reference Doppler power value, as its smooth surface does not have any bubbles trapped on its surface.

A plane-wave Doppler imaging sequence was used, with each parameter tested individually. The digitized signal was monitored to make sure that the analog to digital conversion process had not resulted in a saturated digital signal, since this can also cause a twinkling artifact.

FIG. 4 depicts a table 400 comprising various optimization parameters, in accordance with at least one embodiment. The optimization parameters provided in FIG. 4 are the number of cycles per pulse, the number of pulses per ensemble, the transmit angle, the pulse repetition frequency (RPF), and the Doppler TX voltage.

Data was collected after the Verasonics software beamforming process. The first two pulses in the ensemble were dropped and the remaining pulses were high-pass wall-filtered by a quadratic regression curve fit method. Since the magnitude of the twinkling artifact is required for optimization, Doppler power was calculated for each pixel over the entire imaging plane. The stone and glass sphere positions were then manually selected and the average Doppler power/pixel was calculated for a 5 mm by 5 mm square region centered on the selected target. A 10 mm by 10 mm square region, also centered on the target but excluded pixels from the target, was used as the “noise” value for calculating the effective signal-to-noise ratio (SNR) of the twinkling artifact. The SNR measures how much true signal that is reflecting actual anatomy versus how much noise a particular image has. Three acquisitions were collected for each set of parameters and the SNR of the stone was plotted against the glass sphere as reference.

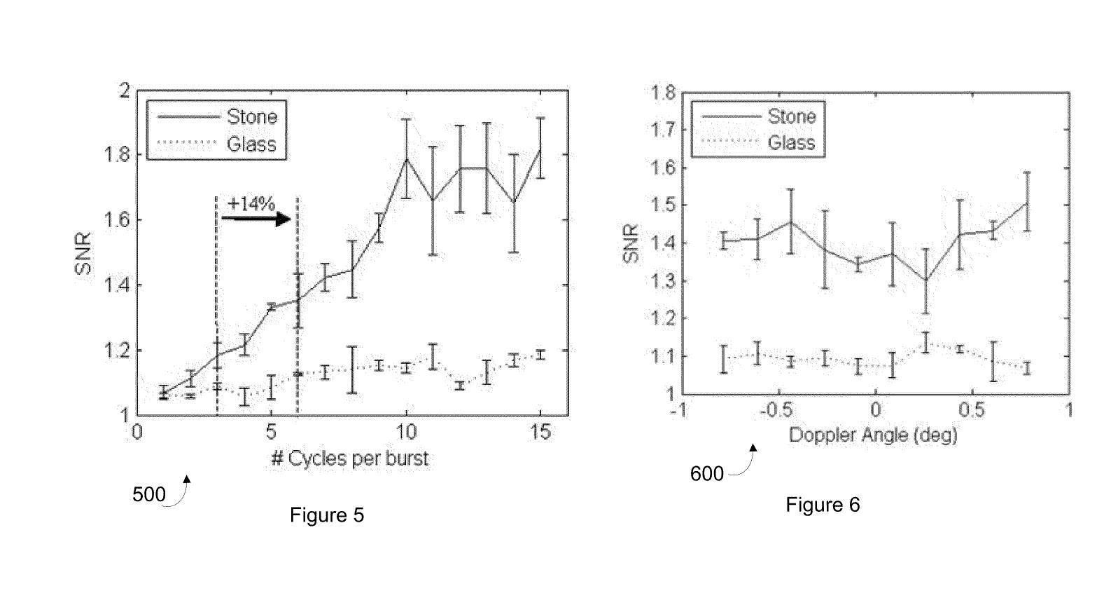

FIG. 5 depicts a graph 500 plotting SNR over number of cycles per burst for both the stone and the glass sphere, in accordance with at least one embodiment. Increasing the number of cycles for each pulse improved the SNR linearly. This effect supports the theory of micron-sized bubbles since increasing the number of cycles of ultrasound generates an increase in random bubble activity. The downside to longer pulses is a decrease in axial resolution, but in this experimental setup twinkling was used to detect the stone and B-mode was used for the actual imaging.

FIG. 6 depicts a graph 600 plotting SNR over a Doppler transmit angle for both the stone and the glass sphere, in accordance with at least one embodiment. As shown in FIG. 6, varying the transmit angle of the Doppler ensemble did not have a significant effect on increasing the SNR. This finding supports the bubble theory because micron sized bubbles should have no angle dependence on their backscatter.

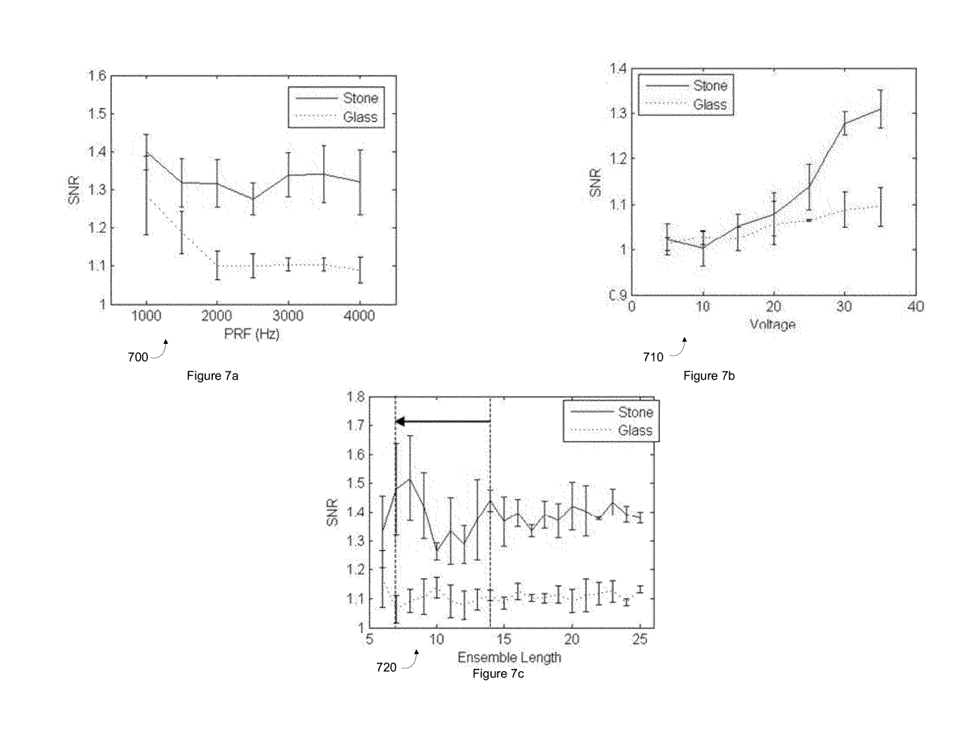

FIG. 7a depicts a graph 700 plotting SNR over Pulse Repetition Frequency (PRF), in accordance with at least one embodiment. As shown in FIG. 7a, SNR remained constant over the tested PRF range. This may be due to the decay time for a micron-sized bubble being much shorter than the period between pulses. Therefore, no pulse should interfere with a prior or subsequent pulse. The independence of PRF on SNR allows for a maximum PRF setting dependent on imaging depth, increasing the range of the velocity measurement, which will improve the efficacy of the wall filter for removing motion artifact and low velocity blood flow.

FIG. 7b depicts a graph 710 plotting SNR over voltage and FIG. 7c depicts a graph 720 plotting SNR over ensemble length, in accordance with at least one embodiment. Increasing the transmit amplitude of the Doppler signal increases the SNR for both the stone and the glass sphere, though the stone has more significant improvement. Ensemble length also did not have an effect on the SNR since the period between pulses is longer than the bubble decay time.

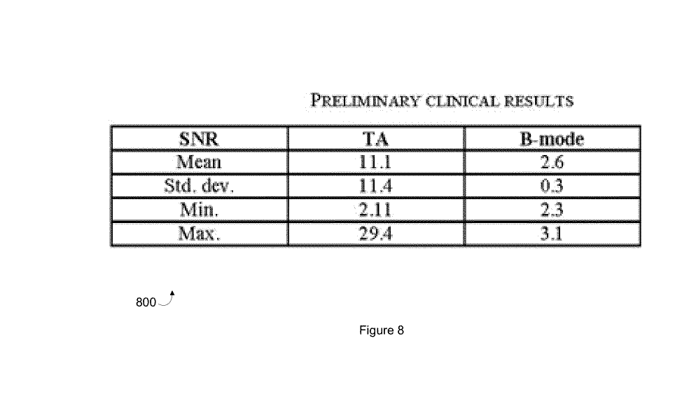

FIG. 8 depicts a table 800 of preliminary clinical results, in accordance with at least one embodiment. A total of five frames of data were collected from two different human patients: two from one patient and three from another, as depicted in table 800. The frame with the lowest SNR was on the same order as the B-mode detection and the maximum SNR was an order of magnitude higher. Therefore, it is suggested that depending on acquisition frame, twinkling artifact has similar if not much better sensitivity compared to B-mode.

An example frame 900 for B-mode and example frame 910 for twinkling artifact are depicted in FIGS. 9a and 9b, in accordance with at least one embodiment.

In another example study, a three-dimensional image was made by collecting ultrasound backscattered data over a two-dimensional region with standard B-mode compression removed. Applying the detection methodologies described herein correctly identified the six subclinical stones (less than 1 mm), while more than six bright spots were observed on the standard B-mode. Thus, applying the methodologies described herein eliminated false positives while detecting all subclinical stones correctly.

As discussed above, the detection of an object in a subject may be used to diagnose, provide a prognosis, monitor treatment and guide treatment decisions for an object, such as a kidney or gall stone, in a body of a subject. The treatment may include medical monitoring or surgical intervention.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims, along with the full scope of equivalents to which such claims are entitled. It is also to be understood that the terminology used herein is for the purpose of describing particular embodiments only, and is not intended to be limiting.

What is claimed is:

1. A method for detecting an object in a body comprising:
transmitting one or more ultrasound pulses to the object and to at least one object of interest on the object;
receiving one or more reflection signals, wherein the one or more reflection signals comprise reflection signals corresponding to the one or more ultrasound pulses reflected from the object and reflection signals corresponding to the one or more ultrasound pulses reflected from the at least one object of interest on the object;
contrasting individual reflection signals from the one or more reflection signals;
displaying a magnitude of interpulse variability;
removing, via a filter, motion signals; and
causing an output device to provide an indication of the presence of the object based on the reflection signals corresponding to the one or more ultrasound pulses reflected from the at least one object of interest on the object.

2. The method of claim 1, further comprising applying color to locations above a threshold brightness on an image that provides the indication of the presence of the object.

3. The method of claim 1, wherein the reflection signals corresponding to the one or more ultrasound pulses reflected from the at least one object of interest comprise variability in phase or amplitude.

4. The method of claim 3, wherein the phase and amplitude between pulses is random and not deterministic as with motion of the target tissue or object.

5. The method of claim 1, wherein artificially amplifying or attenuating at least one of the one or more reflection signals via a filter accentuates variability in phase or amplitude.

6. The method of claim 1, wherein the body is a human body, and wherein the at least one object of interest comprises one or more of the following: a) a bubble, b) a calcification, c) a crevice, d) a crack, e) a concretion, f) a calculus, g) bone, h) a foreign object.

7. The method of claim 1, wherein the object comprises an object selected from the group consisting: a) a kidney stone, b) a gall stone, c) a salivary duct stone, d) a foreign body, e) plaque, f) a vessel, and g) bone.

8. The method of claim 1, wherein the one or more ultrasound pulses are used to excite crevice bubbles on the object to cause the motion of the bubbles to generate the variability among the pulses.

9. The method of claim 8, wherein the one or more ultrasound pulses comprise pulses selected from the group comprising: a) a higher amplitude pulse, b) a longer pulse, c) shorter time between pulses; and d) a low frequency pulse.

10. The method of claim 1, wherein one or more ultrasound pulses comprises two plane wave or flash mode pulses.

11. The method of claim 10, further comprising:
comparing by auto-correlation the two plane wave pulses;
generating a B-mode image from the two plane wave pulses; and
overlaying a color to represent areas of high decorrelation between the pulses on the B-mode image.

12. The method of claim 1, wherein the method is used to diagnose, prognose, or monitor for a kidney stone.

13. A method to diagnose, prognose, or monitor treatment for a kidney stone in a subject, comprising:
transmitting one or more ultrasound pulses to the object and to at least one object of interest on the object;
receiving one or more reflection signals, wherein the one or more reflection signals comprise reflection signals corresponding to the one or more ultrasound pulses reflected from the object and reflection signals corresponding to the one or more ultrasound pulses reflected from the at least one object of interest on the object;
contrasting individual reflection signals from the one or more reflection signals;
displaying a magnitude of interpulse variability;
removing, via a filter, motion signals; and
causing an output device to provide an indication of the presence of the object based on the reflection signals corresponding to the one or more ultrasound pulses reflected from the at least one object of interest on the object; and
diagnosing, prognosing, or monitoring treatment for the kidney stone in the subject based on the size of the object.

14. The method of claim 13, wherein the ultrasound pulses are transmitted in a Doppler ensemble.

15. The method of claim 13, further comprising applying color to locations above a threshold brightness on an image that provides the indication of the presence of the object.

16. The method of claim 13, wherein the reflection signals corresponding to the one or more ultrasound pulses reflected from the at least one object of interest comprise variability in phase or amplitude.

17. The method of claim 13, wherein the phase and amplitude between pulses is random and not deterministic as with motion of the target tissue or object.

18. The method of claim 13, further comprising:
decorrelating the two plane wave pulses;
generating a B-mode image from the two plane wave pulses; and
overlaying the decorrelated pulses on the B-mode image.

19. A computing device, comprising:
a processor; and
a non-transitory computer-readable medium configured to store program instructions thereon executable by the processor to cause the computing device to perform functions comprising:
transmitting one or more ultrasound pulses to the object and to at least one object of interest on the object;
receiving one or more reflection signals, wherein the one or more reflection signals comprise reflection signals corresponding to the one or more ultrasound pulses reflected from the object and reflection signals corresponding to the one or more ultrasound pulses reflected from the at least one object of interest on the object;
contrasting individual reflection signals from the one or more reflection signals;
displaying a magnitude of interpulse variability;
removing, via a filter, motion signals; and
causing an output device to provide an indication of the presence of the object based on the reflection signals corresponding to the one or more ultrasound pulses reflected from the at least one object of interest on the object.

20. The computing device of claim 19, the functions further comprising applying color to locations above a threshold brightness on an image that provides the indication of the presence of the object.
